SEQUENCE LISTING (5 sequences)

SEQ ID NO: 1 - DNA, 61 bp, Homo sapiens
gctgctgccaccgcctgccggccaccagccsgcggccagcaccgcggcgaccgcgcgcggt

SEQ ID NO: 2 - DNA, 61 bp, Homo sapiens
tgaacctgggaggcggagcttgcagtgagcygagatcccgccactgcactccagcctgggc

SEQ ID NO: 3 - DNA, 660 bp, Homo sapiens
aaggtccacgtcaggcggggccccgcccaccgcgggaccaagcggctacgccttccaagttttcattcggggccgcgccagcgctgaggagagtcagacgccagatggttccgaaggtcctgcaggctctcggggcaccctacagccgacacgccaacaaaagcttcggagccgccaaccccaaaacggaaacaccggagccacaagctgagctcgcgtagggcacgctcccccgggagtgcttccgggacagggggcggggtcgccggaagtggccgtccgccgtccgctgctcgttgtttaagcggctgacgggaaggagaagccagagctccagcggcgccgcggggcggcagtcaagaccagagccggagccgtcactcacctctggattagcctgaagcggagactaccggctgcggagcggcggggcgagacacttgctcgccttttgaccccatcatgtcgcggggctccatcgagattcccctccgggacactgacgaggtaagtgtcgtgtatggaggcgggtgggggccatgaacttgtacgatcgcccccaccgacttcgctcgacttcgtttctgaagcgagagcatcctccttccctaagagccctgatcgaaatacctggccaaggtctgatcatcatcttcttca

SEQ ID NO: 4 - DNA, 61 bp, Homo sapiens
caccgcgggaccaagcggctacgccttccargttttcattcggggccgcgccagcgctgag

SEQ ID NO: 5 - DNA, 61 bp, Homo sapiens
tggaggcgggtgggggccatgaacttgtacratcgcccccaccgacttcgctcgacttcgt

(19) United States
(12) Patent Application Publication
(10) Pub. No.: US 2003/0144799 A1
(43) Pub. Date: Jul. 31, 2003

(54) Regulatory single nucleotide polymorphisms and methods therefor

(76) Inventor: Volker Nowotny, Ann Arbor, MI (US)

Correspondence Address: HARNESS, DICKEY, & PIERCE, P.L.C, 7700 BONHOMME, STE 400, ST. LOUIS, MO, 63105, US

(21) Appl. No.: 10/241,151

(22) Filed: Sep. 11, 2002

Related U.S. Application Data
(60) Provisional application No. 60/322,723, filed on Sep. 17, 2001.
(60) Provisional application No. 60/334,543, filed on Nov. 30, 2001.

(51) Int. Cl. C12Q 1/68; G06F 19/00; G01N 33/48; G01N 33/50; C07H 21/04

(52) U.S. Cl. 702/20; 435/6; 536/24.3

(57) ABSTRACT

Polynucleotides containing single nucleotide polymorphisms which are present in transcription factor binding site clusters are disclosed. The polynucleotides can be used in diagnostic assays, in testing systems for finding new drugs, and in methods for treating diseases. Also disclosed are methods for identifying single nucleotide polymorphisms which are present in transcription factor binding site clusters.

### CROSS REFERENCE TO RELATED APPLICATION

This application claims priority to U.S. Provisional Patent Application No. 60/332,723, filed Sep. 17, 2001 and U.S. Provisional Patent Application No. 60/334,543, filed Nov. 30, 2001, both of which are incorporated in their entireties by reference.

### BACKGROUND OF THE INVENTION

(1) Field of the Invention

This invention is related generally to single nucleotide polymorphisms and, more particularly, to single nucleotide polymorphisms (SNPs) associated with regulatory regions for gene expression, i.e. in transcription factor binding site clusters (TFCs) or, more specifically, in transcription factor binding sites. Also included within the invention are polynucleotides containing those SNPs as well as to methods of identifying and methods of using such SNPs in the diagnosis and treatment of disease.

(2) Description of the Related Art

Genomic DNA contains coding sequences, which supply the information for encoding the primary structure of polypeptides, as well as regulatory sequences, which control the amount, the timing, and the cell types in which the polypeptides are synthesized. The regulatory sequences include DNA sequences located at or near transcription start sites where they serve as binding sites for transcription factors. Transcription factors are highly selective for recognition and binding to these binding sites. About 8 contiguous base pares of DNA make up each binding site, although the precise number of base pairs for transcription factor binding domains varies for different transcription factors. The transcription factors selectively bind to their respective binding sites and the amount of a transcription factor present can be tissue or cell-type specific, or can vary in response to developmental or environmental factors.

With the emergence of the genomic sciences, it has become apparent that many diseases involve genetic lesions or mutations. Genetic abnormalities can be as drastic as deletion or abnormal duplication of entire chromosomes (e.g., trisomy 21 or Down's syndrome), or as small as single base pair changes (e.g., the mutation leading to sickle cell disease). The latter group of single base pair sequence differences, whether resulting in phenotypic change or not, are referred to as single nucleotide polymorphisms (SNPs). SNPs represent genomic heterogeneities among individuals in a population. SNPs are ubiquitous among eukaryotic organisms, and may be heritable through the germline, or may result from somatic cell mutation. A nucleotide that is variable (i.e., differs between individuals within a species) is a “SNP site.” Heritable SNP sites are scattered at random throughout the human genome. The human genome contains at least about 3,000,000 SNPs based upon the publicly available genome database and up to as many as about 5,000,000 based upon public and private databases.

Genetic differences such as SNPs can affect the sequence of polypeptides encoded by genes if the SNP lies within the coding region. A defect in primary structure of a polypeptide can be the fundamental cause of a disease. For example, sickle cell hemoglobin results a single amino acid difference from hemoglobin in unaffected individuals. The SNP is, therefore, in this instance the specific cause of the disease.

More commonly, the effects of SNPs can be more subtle. For example, some SNPs are believed to correlate directly with individual predisposition or susceptibility to disease. Correlations between SNPs and diseases can also be indirect, e.g., an SNP can be genetically linked to another mutation that is actually causal for a disease. For many if not most SNPs, no associated phenotype is known.

Several methods have been used to detect SNPs. Human genome databases can provide a basis for initially identifying and investigating the presence of SNPs. Comparison of genomic sequences among individuals of a population can identify SNPs. Once identified, numerous analytical techniques, such as enzyme-based sequencing, polymerase chain reaction (and variations thereof, such as TaqMan® fluorescence release assays) and mass spectroscopy can be used to detect the presence of SNPs in any particular individual.

SNPs can be present in coding and in non-coding regions, including intron regions, splice junctions and regulatory regions. SNPs in regulatory regions, sometimes referred to as regulatory SNPs, have been reported in studies on the association of the SNPs with expression of particular proteins or with disease processes (see for example, Harvey, et al, Brit. J. Haematol. 109:349-353, 2000; Goto et al., Clin. Cancer Res. 7:1952-1956, 2001; Kageyama et al, AIDS Res Hum Retroviruses 17:991-995, 2001; Lynch et al., J. Biol. Chem. 276:24341-24347, 2001; Reynard et al., Eur. J. Immunogenet. 27:241-249, 2000; Keightley et al., Blood 93:427-4283, 1999). Regulatory SNPs have also been incorporated into a database system for analysis of transcription factor binding to target sequences in regulatory gene regions as altered by mutations (Ponomarenko et al., Nucleic Acids Research 29:312-316, 2001). The mutations include naturally occurring regulatory SNPs and site-directed mutations. The database system, referred to as rSNP—Guide, can be accessed on the internet at http://wwwmgs.bionet.nsc.ru/mgs/systems/rsnp/. Nevertheless, the identification of regulatory SNPs has heretofore focused on individual genes or on a small number of genes.

Transcription factor binding sites have been identified by a number of methods (for review see Fickett et al., Curr. Opin. Biotechnol. 11:19-24, 2000). For example, transcription factor binding specificity can be expressed by a consensus sequence. Alternatively, position weight matrix is based upon the generating of a weighted sum for the nucleotides in a particular binding site. Both consensus sequence and position weight matrix have been used in identifying transcription factor binding site matches in genomic DNA (for review, see Frech et al, Trends Biochem Sci. 22:103-104, 1997).

The binding specificities of some transcription factors are known, and at least one database, the TRANSFAC database, has been assembled from verified binding sites. The TRANSFAC transcription factor database is maintained by GBF, Braunschweig, Germany at the internet site of http://transfac.gbf.de/ (Wingender et al., Nucleic Acids Res. 28:298-301, 2001; Wingender et al., Nucleic Acids Res. 28:316-319, 2000). Other databases which include transcriptional regulation sites are COMPL (Kel-Margoulis et al, Nucleic Acids Research 28:311-315, 2000) and TRRD (Kolchanov et al., Nucleic Acids Research 28:298-301, 2000).

Transcription factor binding sites tend to form clusters or TFCs which are sometimes referred to as transcriptional regulatory regions (Fickett et al., supra, 2000). One method for the identification of TFCs is disclosed in U.S. patent application Ser. No. 09/853,141, Publication No. US No. 2002/0037519 A1. The method involves comparing a likelihood parameter that each of a number of protein binding sites will occur in a DNA genomic sequence compared to the likelihood the site will occur in a random nucleotide sequence. The methods described in the '141 patent as well as the consensus sequence and position weight matrix methods provide approaches for identifying a large number of transcription factor binding sites and TFCs.

Nevertheless, high throughput analysis has not been applied to the discovery of a significant proportion of the large numbers of regulatory SNPs which are located in transcription factor binding sites or in TFCs in the human genome

### BRIEF DESCRIPTION OF THE INVENTION

Accordingly, the inventor herein has succeeded in devising an approach which has identified a large number of regulatory SNPs located in transcription factor binding sites and in TFCs. Reference to TFCs herein is intended to mean the 5′ to 3′ sequence of DNA which contains two or more transcription factor binding sites. The SNPs are identified by comparing SNP-containing nucleotide sequences from SNP databases with transcription factor binding site sequences and TFC sequences. SNP-containing nucleotide sequences can be found in databases such as the publicly available dbSNP database maintained by the National Center for Biotechnology Information available on the internet at http://www.ncbi.nlm.nih.gov/SNP/. The SNPs are identified as the observed variant bases within a longer sequence of bases. Transcription factor binding site sequences can be found in the art such as in the TRANSFAC database of transcription factor binding site sequences. TFC sequences can be identified by any of a number of methods, and, in particular, using the methods disclosed in U.S. patent application Ser. No. 09/853,141.

Regulatory SNPs are identified by comparing the genomic sequences surrounding the SNP with genomic sequences including and surrounding the TFCs or transcription factor binding sites. Thus, a SNP-containing sequence is determined to be a regulatory SNP sequence if the SNP-containing sequence maps to a TFC sequence or, in a more narrow set of regulatory SNP sequences, if the SNP-containing sequence maps to a transcription factor binding site sequence. The particular length of flanking sequence surrounding the SNP and the flanking sequence surrounding a TFC or transcription factor binding site, will depend upon the degree of certainty desired in assembling the set of regulatory SNP sequences. The use of short flanking sequences of 10 to 20 nucleotides for the comparison will generate a larger number of putative regulatory SNP sequences many of which are false positives, i.e. SNPs which do not lie within a TFC or a transcription factor binding site. Preferably, at least about 30 nucleotides of flanking sequences is used such that the genomic SNP-containing sequences being tested to determine whether they are regulatory SNP sequences, will be about 61 nucleotides in length, including the SNP, about 30 nucleotides 5′ to the SNP and about 30 nucleotides 3′ to the SNP. Similarly, the genomic sequences containing the transcription factor binding sites, which can be a TFC sequences or a transcription factor binding site sequences, will also include about 30 nucleotides 5′ and about 30 nucleotides 3′ to the TFC sequences or transcription factor binding site sequences. As a result, regulatory SNPs identified will lie within a region from the first 5′ nucleotide to the last 3′ nucleotide of the TFC or transcription factor binding site

The regulatory SNP sequences identified form the basis for a set of regulatory SNP polynucleotides which correspond to the regulatory SNP sequences. Thus, the regulatory SNP polynucleotides comprise 5′ flanking sequence, 3′ flanking sequence or both 5′ and 3′ flanking sequences which are identical to genomic sequences surrounding the SNP. The regulatory SNP polynucleotides comprise at least 6 contiguous nucleotides which include the SNP and flanking sequence, preferably at least 10 contiguous nucleotides, preferably at least 15 contiguous nucleotides, preferably at least 20 contiguous nucleotides, preferably at least 30 nucleotides or more.

Unless otherwise indicated, reference to polynucleotide, sequence or polynucleotide sequence within the present invention is intended to include the complementary polynucleotide, sequence or polynucleotide sequence as well. It is preferred that when a particular polynucleotide is included in a set of regulatory SNP polynucleotides, its complement will not be included. In sets in which polynucleotides which are complementary are included within a given set, the complementary polynucleotides are, in some instances, considered one member of the set.

Since either of the two variant bases could be considered to be associated with a disease or condition either in a positive or in a negative manner, both variants are considered to be included within the database of regulatory SNPs as well as within the set of regulatory SNP polynucleotides. For example, whereas one regulatory SNP present in a minority of the population might be considered directly related to the disease, another regulatory SNP present in a minority of the population could confer a disease resistance absent in the majority such that the SNP in the majority could be considered more directly linked to the disease. Furthermore, when testing for presence or absence of a particular regulatory SNP variation, either variant could potentially be used if only the two variations exist in the population as a whole. Thus, the databases of regulatory SNPs are, thus, contemplated as including both variants as one member of a given database. Correspondingly, the sets of regulatory SNP polynucleotides of the present invention can, thus, be considered to include two regulatory SNP polynucleotide variations for a given SNP as one member of the set. In such instances where a third or fourth variation could be present, the three or four regulatory SNP polynucleotide variations are considered as one member of the set.

Thus, in one aspect, the present invention is directed to a set of regulatory SNP polynucleotides or a set of polynucleotides complementary thereto. The set of regulatory SNP polynucleotides comprise a plurality of polynucleotides, each having at least 6 contiguous nucleotides. Each polynucleotide of the set comprises a regulatory SNP with 5′, 3′ or both 5′ and 3′ genomic flanking sequence. While each regulatory SNP polynucleotide will have one SNP, at least two of the regulatory SNP polynucleotides will each have a different SNP. Thus, the set of regulatory SNP polynucleotides comprises a plurality of regulatory SNPs and this plurality of regulatory SNPs collectively map to a plurality of TFC sequences. At least one of the regulatory SNPs will map to at least one TFC and at least one other regulatory SNP will map to at least one other TFC. By map or mapping it is meant that the SNP is present at a specific location in the genome which can be identified by the specific flanking sequence immediately adjacent to each SNP. Thus, each SNP lies within a TFC sequence and this can be verified by determining that the genomic nucleic acid sequence from 30 nucleotides 5′ to 30 nucleotides 3′ to each SNP is identical or complementary except for the SNP, to a portion of a genomic nucleic acid sequence from 30 nucleotides 5′ to 30 nucleotides 3′ to the TFC sequence. Reference to a genomic nucleic acid sequence containing the SNP and flanking sequence is intended to mean a genomic sequence in which the SNP has been identified, whether that genomic sequence is the full length genomic sequence of a chromosome or a portion thereof. Typically SNPs are identified in databases with less than 1 kb of flanking sequence.

In another aspect, the set can be more narrowly defined such the set comprises regulatory SNP polynucleotides in which the SNPs map to transcription factor binding sites. Thus, a SNP would be determined to be a regulatory SNP if a genomic sequence from 30 nucleotides 5′ to 30 nucleotides 3′ of said each SNP is identical or complementary except for the SNP, to a portion of a genomic nucleic acid sequence from 30 nucleotides 5′ to 30′ nucleotides 3′ to said transcription factor binding site sequence.

In still another aspect, the set can be more broadly defined such that the set comprises SNP polynucleotides in which the SNPs map to a longer genomic sequence from 100 nucleotides 5′ to 100 nucleotides 3′ to TFC sequences. Thus, a SNP would be determined to be a regulatory SNP if a genomic sequence from 30 nucleotides 5′ to 30 nucleotides 3′ of said each SNP is identical or complementary except for the SNP, to a portion of a genomic nucleic acid sequence from 130 nucleotides 5′ to 130 nucleotides 3′ to one of the TFC sequence.

The set of polynucleotides can comprise a set of probes or primers which can be placed on one or more biochips. The probes or primers on the biochip can be those which have been identified as being associated with a particular disease or condition. Reference to disease herein is intended to mean a pathological state of the body that presents a group of clinical signs and symptoms as well as laboratory findings. Also included are illness and suffering which may or may not arise from pathological changes in the body. By condition, reference is made to a physical attribute or state of functioning of the body which are usually not directly associated with pathological states of the body.

The present invention also includes methods for diagnosing presence of a disease or predisposition for developing a disease associated with a regulatory SNP. The methods involve detecting the presence of the regulatory SNP in an individual.

As used herein, the term individual is intended to refer primarily to human subjects or patients although non-human mammalian subjects or patients are within the scope of the present invention. Where applicable to individuals of a non-human mammalian species, the present invention is directed to regulatory SNPs, to regulatory SNP polynucleotides and to methods therefor in which the SNPs are present in the genomes of at least some of the members of the non-human mammalian species.

Also included within the present invention are methods for identifying a substance for treating a disease associated with a regulatory SNP. Such methods involve testing compounds for activity in advantageously modulating gene-product expression altered by a regulatory SNP.

The present invention also includes methods for treating or preventing a disease associated with a regulatory SNP. The methods involve modulating gene product expression to approach expression in individuals not having the regulatory SNP.

Methods of identifying regulatory SNPs are also within the scope of the present invention. The methods comprise determining that a SNP is a regulatory SNP if the SNP maps to a TFC. By mapping to a TFC, it is meant that the SNP lies within a TFC sequence and a genomic nucleic acid sequence from at least 20 nucleotides 5′ to at least 20 nucleotides 3′ to said candidate SNP is identical or complementary except for the SNP, to a portion of a genomic nucleic acid sequence from at least 20 nucleotides 5′ to at least 20 nucleotides 3′ to the TFC sequence. More preferably, the SNP is determined to be a regulatory SNP if a genomic nucleic acid sequence from at least 30 nucleotides 5′ to at least 30 nucleotides 3′ to said candidate SNP is identical or complementary except for the SNP, to a portion of a genomic nucleic acid sequence from at least 30 nucleotides 5′ to at least 30 nucleotides 3′ to the TFC sequence.

The methods of identifying regulatory SNPs also include determining that a SNP is a regulatory SNP if the SNP maps to a transcription factor binding site. The SNPs of this aspect of the present invention lie within transcription factor binding sites sequence and a genomic nucleic acid sequence from at least 20 nucleotides 5′ to at least 20 nucleotides 3′ to said candidate SNP is identical or complementary except for the SNP, to a portion of a genomic nucleic acid sequence from at least 20 nucleotides 5′ to at least 20 nucleotides 3′ to the transcription factor binding site sequence. More preferably, the SNP is determined to be a regulatory SNP if a genomic nucleic acid sequence from at least 30 nucleotides 5′ to at least 30 nucleotides 3′ to said candidate SNP is identical or complementary except for the SNP, to a portion of a genomic nucleic acid sequence from at least 30 nucleotides 5′ to at least 30 nucleotides 3′ to the transcription factor binding site sequence.

The methods of identifying regulatory SNPs can also be more broadly defined to include determining that a SNP is a regulatory SNP if the SNP maps to a longer genomic sequence from 100 nucleotides 5′ to 100 nucleotides 3′ to TFC sequences. Thus, a SNP would be determined to be a regulatory SNP if a genomic sequence from 20 nucleotides 5′ to 20 nucleotides 3′ of said each SNP is identical or complementary except for the SNP, to a portion of a genomic nucleic acid sequence from 120 nucleotides 5′ to 120 nucleotides 3′ to one of the TFC sequences. More preferably, the SNP is determined to be a regulatory SNP if a genomic sequence from 30 nucleotides 5′ to 30 nucleotides 3′ of said each SNP is identical or complementary except for the SNP, to a portion of a genomic nucleic acid sequence from 130 nucleotides 5′ to 130 nucleotides 3′ to one of the TFC sequences.

Also included in the present invention is a computer readable medium having a data structure for use in reporting regulatory SNPs. The data structure comprises a first field containing either or both of sequence and genomic mapping location sequence information on SNPs; a second field containing either or both of sequence and genomic mapping location of TFCs; and a third field containing information on regulatory SNPs. The regulatory SNPs are determined and reported by the identities of either or both of sequences and genomic mapping locations of SNPs and TFCs.

### BRIEF DESCRIPTION OF THE DRAWINGS

FIG. 1 illustrates the hypothetical relationship of SNPs to a TFC containing transcription factor binding sites, TF-A, TF-B, TF-C and TF-D.

FIG. 2 illustrates the relationship of a TFC shown in capital letters with transcription factor binding sites within the TFC identified by boxes and named within each box, along with two associated regulatory SNPs shown above the TFC with 30 nucleotides 5′ and 30 nucleotides 3′ in small letters and the SNP capitalized and bolded.

### DETAILED DESCRIPTION OF THE INVENTION

In accordance with the present invention, methods for identifying regulatory SNPs occurring in TFCs and in transcription factor binding site sequences are provided. The methods have generated a database of regulatory SNPs and have provided the basis for the sets of regulatory SNP polynucleotides of the present invention. The term “set” is used herein interchangeably with the term “collection”. The regulatory SNP polynucleotides are relatively short polynucleotides which comprise the regulatory SNP along with genomic flanking sequence. The set of polynucleotides or collection of polynucleotides are such that each polynucleotide is of a length suitable for selective hybridization to the genomic sequence containing the SNP. Such selective hybridization can form the basis for methods of detecting the presence of the SNP in a genomic DNA sample from an individual. The regulatory SNP database and the set of regulatory SNP polynucleotides can also be generated by other approaches such as, for example, by validating effects of SNPs one-by-one on transcription factor binding and gene expression. However, the approach described herein has allowed the construction of a database of a large number of regulatory SNPs and corresponding regulatory SNP polynucleotides.

By selective hybridization or specific hybridization it is meant that a polynucleotide preferentially hybridizes with a target sequence. Typically such selective or specific hybridization is carried out under high stringency conditions. Suitable stringency conditions can be selected by the skilled artisan on the basis of factors known to control the stringency during hybridization and during the washing procedure, including temperature, ionic strength, length of time, and concentration of formamide (for reference, see Sambrook, et al, Molecular Cloning: A Laboratory Manual, Cold Spring Harbor Laboratory Press, Cold Spring Harbor, N.Y., 1989).

The polynucleotide length suitable for selective hybridization to the genomic sequence will also depend upon hybridization conditions as well as conditions specific to the particular testing procedure. Preferably the polynucleotide will be comprised of at least 6 contiguous nucleotides which include the SNP and genomic flanking sequence, more preferably at least 10 contiguous nucleotides, more preferably at least 15 contiguous nucleotides, more preferably at least 20 contiguous nucleotides, more preferably at least 30 nucleotides or more.

The database of regulatory SNPs and the collection of regulatory SNP polynucleotides can be small having at least 2, at least 3, at least 5, at least 10 or at least 20 SNPs or SNP polynucleotides, however, larger numbers of SNPs or SNP polynucleotides are more preferred because they provide a more comprehensive coverage of the genome. As such the database of SNPs or the set of regulatory SNP polynucleotides preferably, has at least about 100, at least about 200, at least about 1000, at least about 10,000 or more members.

Furthermore, the individual members of the database of regulatory SNPs and the individual members of the set of regulatory SNP polynucleotides are substantially all regulatory SNPs or regulatory SNP polynucleotides, respectively. Regulatory SNPs as used herein is intended to refer to SNPs located in transcription factor binding sites or more broadly, SNPs located in TFCs and still more broadly, SNPs located within a region from 100 nucleotides 5′ to 100 nucleotides 3′ to the TFC. It is also understood by the skilled artisan that it is possible that certain transcription factor binding sites identified may not have a meaningful in vivo function because other factors such as competition, chromatin structure and other influences could be more important than binding affinity of a transcription factor to the site (see Fickett et al., 2000, supra; Audic et al., Trends Genet. 14:10-11, 1998). Correspondingly, it is possible that certain regulatory SNPs within the database may not have a meaningful in vivo effect.

The polynucleotides of the present invention are suitable for use as probes and primers for detecting the regulatory SNPs in particular on one or more biochips. A microarray of the polynucleotides are placed on a solid support to form the biochip and specific hybridization of a genomic DNA sample from an individual is detected. Such methods can be used to detect presence of the SNP. One particularly preferred aspect is a microarray of regulatory SNP-containing probes or primers known to be associated with a particular disease. Such a collection of probes or primers is generated based upon studies in which a set of regulatory SNPs is determined to be present in a population of individuals having a particular disease compared to normal individuals, i.e. individuals not having the disease. Every individual among the population of individuals having the disease need not have all of the regulatory SNPs, however, the population of individuals with the disease collectively show the presence of all of the set of regulatory SNPs such that association with the disease is established.

The set of probes or primers assembled on a biochip in this particular aspect of the present invention, are substantially all regulatory SNP polynucleotides associated with a particular disease. The number of regulatory SNP polynucleotides on the biochip is sufficiently large to provide a meaningful assessment of the collective regulatory effect of the SNPs on genes whose expression is associated with the particular disease. Additionally, the number of regulatory SNP polynucleotides on the biochip and associated with the particular disease, is substantially smaller than the overall number of regulatory SNPs and regulatory SNP polynucleotides of the present invention. The number of regulatory SNPs on the biochip of this aspect of the invention can be from about 5 to about 5,000 or greater and, preferably, at least about 5, at least about 10, at least about 15, at least about 20, at least about 30, at least about 50, at least about 100, at least about 200, at least about 1000 or greater. It is understood by the skilled artisan that the number of genes associated with a particular disease and, hence, the number of regulatory SNP polynucleotides associated with that disease, will depend upon the particular disease. It is also to be understood that whereas a very large number of genes could be associated in some minor way with a particular disease, only a somewhat smaller number of genes and SNP polynucleotides would be expected to show a meaningful association with the disease.

Any of a number of detection protocols for detecting the regulatory SNPs can be used such as, for example, electrophoresis-based genotyping methods, fluorescence-based genotyping and mass spectrometry based detection. Regulatory SNPs can be identified by any of a number of methods, including single nucleotide primer extension, allele-specific hybridization or primer extension, oligonucleotide ligation assay and invasive signal amplification (for reviews, see Shi, Clin. Chem. 47:164-172, 2001; Nowotny et al., Curr. Opin. Neurobiol. 11:637-641, 2001; Kwok, Annu. Rev. Genomics Hum. Genet. 2:235-258, 2001; Brennan, Am. J. Pharmacogenomics 1:295-302).

Methods of the present invention can be used to generate a database of regulatory SNPs which has served as a basis for generating the various sets of regulatory SNP polynucleotides of the present invention. The methods are based upon comparison of sequences flanking SNPs with TFC sequences and sequences of transcription factor binding sites. This is accomplished by using a SNP database from which 5′ and 3′ flanking sequences are identified and compared with sequences in a database of TFCs sequences of transcription factor binding site sequences. This comparison can be a direct comparison of sequence identities or an indirect comparison in which the genomic location of a particular SNP is determined and compared to the genomic location of the transcription factor binding site. The direct comparison process is efficiently performed using computer software suitable for comparing sequences and identifying perfect matches of 5′ and 3′ nucleotides flanking the SNP. Preferably, 20 nucleotides on either side of the SNP are used, i.e. a sequence of 41 nucleotides in which the SNP lies at position 21 and, more preferably, 30 nucleotides on either side of the SNP are used, i.e. a sequence of 61 nucleotides in which the SNP lies at position 31. All of the nucleotides of the sequence, for example 41 or 61 nucleotides, except for the SNP, are required to match the sequence of a TFC sequence or to a transcription factor binding site sequence, whereupon, the computer program reports the finding of concordance of sequences. Such sequence identity indicates that the SNP lies within a TFC sequence or within a transcription factor binding site sequence and, hence, the SNP is a regulatory SNP. Sequences of other lengths (shorter or longer) can also be used, for example a sequence extending 10 nucleotides 5′ to the regulatory SNP to 10 nucleotides 3′ to the regulatory SNP, or 100 nucleotides 5′ to the regulatory SNP to 100 nucleotides 3′ to the regulatory SNP.

Regulatory SNPs making up the database of SNPs can be identified by Accession number as well as by sequence based upon the publicly available dbSNP database maintained by the National Center for Biotechnology Information and accessible on the internet at http://www.ncbi.nlm.nih.gov/SNP/.

Databases containing transcription factor binding sites are also available such as, for example the TRANSFAC transcription factor database maintained by GBF noted above. Furthermore, TFCs can be identified by the method disclosed in U.S. patent application Ser. No. 09/853,141, Publication No. US 2002/0037519 A1. The method involves comparing a likelihood parameter that each of a number of protein binding sites will occur in a DNA genomic sequence compared to the likelihood the site will occur in a random nucleotide sequence. The TFCs which are generally located 5′ to the transcription start site of genes, comprise a plurality of transcription factor binding sites which can act in concert to control gene expression. Using the sequence comparison approach of the present invention, a large number of regulatory SNPs have been identified.

The databases of SNPs generated by the methods of the present invention can be used to correlate presence of the SNP with gene expression. As such, the database of SNPs can be a group of SNPs associated with expression of gene-products or associated with a particular set of gene products such as, for example, the expression of cytokines. Sets of regulatory SNP polynucleotides can also be generated corresponding to such SNPs

Databases of SNPs can also be selected on the basis of the SNPs in the group being associated with a particular disease or condition. Because disease states can involve alterations in gene expression, the presence of certain SNPs in an individual can correlate with the presence of or predisposition or susceptibility to disease. Thus, SNPs found in sequences where transcription factors and other regulatory proteins normally bind can be associated with altered transcription factor binding, thereby altering gene expression and contributing to a diseased state. The collective effects of a plurality of regulatory SNPs on the expression of a plurality of genes can produce overt changes in phenotypes, including certain diseases and conditions.

The database of regulatory SNPs as well as the collection of regulatory SNP polynucleotides of the present invention associated with a particular disease, can be small including at least 2, at least 3, at least 5, at least 10 or at least 20 SNPs or SNP polynucleotides, however, larger numbers of SNPs or SNP polynucleotides may be involved with a particular disease. Such larger databases of SNPs or sets of regulatory SNP polynucleotides may contain at least about 100, at least about 200, at least about 1000, at least about 10,000 or more members.

The regulatory SNP polynucleotides provide the basis for methods for dealing with diseases and conditions associated with the gene expression. Such methods can involve diagnostic approaches for detecting a disease or condition or a predisposition to a disease or condition in an individual. The diagnostic methods of the present invention can involve the determining of the presence of a disease or condition, the estimation of prognosis or probable outcome of the disease or condition and prospect for recovery from the disease or the prospect of ameliorating or modifying the condition, the monitoring of the status of the disease or condition or the recurrence of the disease or condition, and the determining of a preferred therapeutic regimen or ameliorative actions for the individual.

Methods of diagnosis can be based upon detection of the presence of regulatory SNPs associated with the disease or condition. Such detection can be based upon various hybridization methods as discussed above and, in particular, the use of biochips which comprise a set of regulator SNP polynucleotides. The regulatory SNP polynucleotides on the biochips are probes or primers specific for the SNPs associated with the disease or condition.

The methods can also involve treatment of a disease or condition or preventing the occurrence of a disease or condition in which the disease or condition is associated with a regulatory SNP. The method comprises administering to an individual in need thereof, a substance which modulates gene-product expression. Both gene-product expression and the presence of a regulatory SNP are associated with presence of or predisposition to developing the disease or condition. The treatment can involve modulation of gene expression at any level of control and preferably modulation at the level of transcription factors binding to transcription factor binding sites. Such treatment can involve the use of antisense molecules in such instances in which a gene product is underexpressed and the transcription factor binding site is a repressor site and also in such instances in which a gene product is overexpressed and the transcription factor binding site is an enhancer site.

The antisense oligonucleotides have nucleotide sequences that interact through base pairing with a specific complementary nucleic acid target sequences which are transcription factor binding sites. The term complementary to a nucleotide sequence in the context of antisense oligonucleotides means sufficiently complementary to the target sequence as to allow hybridization to that sequence in a cell under physiological conditions. Antisense oligonucleotides preferably comprise a sequence containing from about 8 to about 100 nucleotides and more preferably, from about 15 to about 30 nucleotides. Antisense oligonucleotides can also include derivatives which contain a variety of modifications that confer resistance to nucleolytic degradation such as, for example, modified internucleoside linkages modified nucleic acid bases and/or sugars and the like (Uhlmann and Peyman, Chemical Reviews 90:543-584, 1990;-Schneider and Banner, Tetrahedron Lett 31:335, 1990; Milligan et al., J Med Chem 36:1923-1937, 1993; Tseng et al., Cancer Gene Therap 1:65-71, 1994; Miller et al., Parasitology 10:92-97, 1994 which are incorporated by reference). Such derivatives include but are not limited to backbone modifications such as phosphotriester, phosphorothioate, methylphosphonate, phosphoramidate, phosphorodithioate and formacetal as well as morpholino, peptide nucleic acid analogue and dithioate repeating units.

The treatment can also involve modulation of gene expression by administration of a recombinant DNA encoding a transcription factor. The recombinant DNA preferably includes a eukaryotic promoter for expression of the transcription factor. In such instances in which the gene product is overexpressed, the recombinant transcription factor can be selected to be one that binds to a repressor site and in such instances in which the gene product is underexpressed, the recombinant transcription factor can be selected to be one that binds to an enhancer site.

The present also includes a method for identifying a substance for treating a disease or condition. Both gene-product expression and the presence of a regulatory SNP are associated with presence of or predisposition to developing the disease or condition. The method involves testing a candidate compound for activity in modulating gene expression at any level of control and preferably modulation at the level of transcription factors binding to transcription factor binding sites. Such compounds can involve antisense molecules in such instances where a gene is overexpressed and the transcription factor binding site is an enhancer site and also in such instances where a gene product is underexpressed and the transcription factor binding site is a repressor site.

The substances can also be a recombinant DNA which encodes a transcription factor. The recombinant DNA preferably includes a eukaryotic promoter for expression of the transcription factor. In such instances in which the gene product is overexpressed, the recombinant transcription factor can be selected to be one that binds to a repressor site and in such instances in which the gene product is underexpressed, the recombinant transcription factor can be selected to be one that binds to an enhancer site.

The regulatory SNP database of the present invention can be used in establishing correlations between regulatory SNPs and disease to form the basis for diagnostic, treatment and assay methods above. In order to establish this correlation, SNP data is collected from diseased individuals and compared to that of normal individuals. Such data collection involves obtaining nucleic acid samples from the individuals having a particular disease. Once the nucleic acid samples are obtained, the next step would be to determine which regulatory SNPs are present in the sample. Any of a number of methods can be used for detecting regulatory SNPs based upon standard methodology used in molecular biology (for example, see Sambrook, et al, Molecular Cloning: A Laboratory Manual, Cold Spring Harbor Laboratory Press, Cold Spring Harbor, N.Y., 1989). For example, nucleic acid detection methods used to detect SNPs can also be used to detect regulatory SNPs (see for example U.S. Pat. Nos. 6,171,785, 5,945,283, 5,210,015, and 5,487,972). Examples of such methods include the TaqMan® fluorescence release assays (Applied Biosystems, Foster City, Calif. 94404), dideoxynucleotide incorporation assays, biochip-based assays and mass spectroscopy.

By gathering regulatory SNP data from a population of individuals using appropriate epidemiological and statistical methods, the presence of particular regulatory SNPs can be correlated with the presence, susceptibility, or predisposition to a disease or condition.

In most instances, such correlations with a particular disease will not be limited to a single regulatory SNP, a single transcription factor binding site or a single TFC. Instead, families of transcription factor binding sites and families of TFCs are believed to correlate with most diseases or conditions. In a hypothetical example, if it is determined that a population of individuals having a disease or condition or predisposition to a disease or condition collectively have 10 regulatory SNPs, then such a correlation establishes a useful molecular tool for diagnosing the disease as well as targets for developing new drug candidates and for treating individuals with the disease or condition.

Diagnostic databases can be used to test individuals for the presence of regulatory SNPs correlating with the presence of or susceptibility to a disease. DNA analysis to determine the presence of regulatory SNPs can be performed using any standard method of DNA analysis as discussed above. In this way, a regulatory SNP profile for a patient can be generated with the aid of regulatory SNP polynucleotides. The polynucleotides can be probes or primers. The collection of probes can be disease-specific such that each of the probes specifically hybridizes to a transcription factor binding site containing a regulatory SNP which has been identified as being related to a disease. By probe or primer, reference is made to a molecule which has affinity and binding specificity for a predefined nucleic acid target site. Typically, a probe or primer comprises a sequence of at least about 6 nucleotides or base pairs. Preferably, a probe or primer comprises a sequence of at least about 10, at least about 15, at least about 20, at least about 25, at least about 50 or greater nucleotides. The probes are useful in methods for detection of SNPs by virtue of their binding to a target nucleic acid sequence associated with a disease (for examples of such methods, see U.S. Pat. Nos. 6,171,785, 5,945,283, 5,210,015, and 5,487,972). Probes can be detected using labels, for example by comprising radioisotopes or fluorochromes during chemical synthesis of the probe. The regulatory polynucleotides can also be primers for elongation of DNA, i.e., new synthesis commencing at the 3′ end of the primer. The probes or primers hybridize, i.e., form specific base-pairing duplexes with complementary sequences.

One method for analyzing a DNA sample for the nucleotide composition of a SNP site, involves use of isolated nucleic acid molecules in conjunction with dideoxy terminators. In accordance with this method, an oligonucleotide probe of about 20 base pairs in length comprising sequences immediately adjacent 5′ to a regulatory SNP is allowed to hybridize with a sample of a subject DNA using a denaturing/renaturing (e.g., heating and cooling) cycle. Components of a reaction mix comprising DNA polymerase and all four standard dideoxynucleotide triphosphates (ddNTP's, each ddNTP further comprising a distinct covalently bound fluorochrome or other detectable label) are added, and an elongation reaction is allowed to proceed. The reaction can also proceed if the steps are conducted in order of adding the ddNTP's and a DNA polymerase prior to a denaturation/renaturation heating cycle if a thermostable DNA polymerase such as taq polymerase is used. In the denaturation/renaturation cycle, the oligonucleotide hybridizes with the patient's DNA immediately 5′ to a regulatory SNP site. Because ddNTP's terminate elongation reactions, only a single dideoxynucleotide will then add on to the 3′ end of oligonucleotide primers that have hybridized with the patient's DNA. The newly incorporated oligonucleotide will be the base-pairing partner of the SNP site. Following separation of elongated primer from unincorporated dideoxynucleotides using standard methods, the dideoxynucleotide incorporated at the 3′ end of the oligonucleotide is easily determined using a fluorometer, a fluorescence microscope, a spectrophotometer, or other means for determining the species of label. If mass spectroscopy analysis is available, such as matrix assisted laser deabsorption time of flight, mass spectroscopy, ddNTP's without any added label can be used and the incorporated nucleotide determined (see, for example, Crain et al., Current Opinion in Biotechnology 9, 25-34, 1998). Because adenine forms a base pair with thymine, and guanine forms a base pair with cytosine, the nucleotide comprising the regulatory SNP site is easily determined as the base-pairing partner of the incorporated nucleotide. By using a library of such oligonucleotides, as many individual reactions can be set up as needed to generate a regulatory SNP profile on a patient.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

This example illustrates the generation of a database of transcription factor binding site clusters.

A search algorithm for transcription factor binding site clusters, tfblast as disclosed in U.S. patent application Ser. No. 60/203,469 (which is incorporated by reference in its entirety) was used. This algorithm generates transcription factor binding site clusters using the TRANSFAC database maintained by GBF at the internet site of http://transfac.gbf.de/. This was executed against the Genbank human genome database which is accessible at the internet site http://www.ncbi.nlm.hig.gov/Genbank/index.html. Application of the search algorithm identified clusters of transcription factor binding sites, the start points and end points of the clusters, and the positions of the clusters within the human genome. A total of approximately 58,000 transcription factor binding site clusters were identified and collected into a database (the “TFCdb” database).

### EXAMPLE 2

This example illustrates the identification and mapping of regulatory SNPs.

A database of SNPs mapped to the human genome was constructed using the publicly available dbSNP database maintained by the National Center for Biotechnology Information available on the internet at http://www.ncbi.nlm.nih.gov/SNP/. Data downloaded from the SNP database was shortened to sequence strings of 61 nucleotides, each comprising 30 nucleotides 5′ to a SNP, a SNP, and 30 nucleotides 3′ to a SNP. For example, the sequences with accession number ss100070 and ss1000934 were shortened to the 61 base sequences as follows

| | |
|---|---|
| ss100070: | (SEQ ID NO:1) |

GCTGCTGCCACCGCCTGCCGGCCACCAGCC(S)GCGGCCAGCACCGCGGC
GACCGCGCGCGGT

| | |
|---|---|
| ss1000934: | (SEQ ID NO:2) |

TGAACCTGGGAGGCGGAGCTTGCAGTGAGC(Y)GAGATCCCGCCACTGC
ACTCCAGCCTGGGC,

where the parentheses indicate the SNP, and “S” represents a G or C, in accordance with WIPO Standard ST.25 for nucleotides, wherein “A” represents adenine; “G” represents guanine; “C” represents cytosine; “T” represents thymine; “U” represents uracil; “R” represents guanine or adenine; “Y” represents thymine/uracil or cytosine; “M” represents adenine or cytosine; “K” represents guanine or thymine/uracil; “S” represents guanine or cytosine; “W” represents adenine or thymine/uracil; “B” represents guanine, cytosine, or thymine/uracil; “D” represents adenine, guanine, or thymine/uracil; “H” represents adenine, cytosine or thymine/uracil; “V” represents adenine, cytosine, or thymine/uracil; and “N” represents adenine, guanine, cytosine, thymine/uracil, unknown, or other. Letters as used herein for base representations can interchangeably be either small or capital letters.

About 4,600,000 such sequences were collected as entries in a data set. The number of entries was then reduced by elimination of multiple occurrences of identical sequences (ignoring the SNP) and by removing sequences that were stored in the dbSNP database but were described as having no polymorphism. The number of entries in the data set was further reduced by detecting and eliminating identical sequences revealed by examination of sequence complements. These steps reduced the data set to about 2,300,000 sequences.

Although mapping SNPs to the human genome could then, in principle, be accomplished through direct comparison of the SNP sequences in the data set to the approximately 3,000,000,000 nucleotides in a human genome sequence database, base-by-base determination of sequence identity would require approximately 2×10¹⁷ comparisons. Because such an effort is impractical, an algorithm was developed that reduced the amount of calculations to a practical level. In this algorithm, the base content of each 60 base sequence (and its complement) flanking a SNP was calculated as used as a filter. Using the 61 base sequence of accession number ss100070, GCTGCTGCCACCGCCTGCCGGCCACCAGCC(S)GCGGCCAGCACCGCGGCGAC CGCGCGCGGT (SEQ ID NO:1) and accession number ss1000934, TGAACCTGGGAGGCGGAGCTTGCAGTGAGC(Y)GAGATCCCGCCACTGCACTC CAGCCTGGGC (SEQ ID NO:2), ss10070 is made up of 35% G, 48% C, 10% A, and 7% T (ignoring the SNP) and its complement comprises 35% C, 48% G, 10% T, and 7% A (ignoring the SNP); ss1000934 is made up of 35% G, 32% C, 17% A, and 15% T (ignoring the SNP) and its complement comprises 35% C, 32% G, 17% T, and 15% A (ignoring the SNP). The calculated base compositions of the 60 base SNP-flanking sequences were then compared against the human genome sequence by calculating the base composition of all 61 base (minus the SNP) sequences of the human genome. Only sequences with identical base composition were used in a follow-up base-by-base comparison. Base-by-base comparisons were terminated whenever a mismatch was encountered. All completely successful comparisons were captured in a table within the TFCdb database. In this example, GCTGCTGCCACCGCCTGCCGGCCACCAGCC(S)GCGGCCAGCACCGCGGCGAC CGCGCGCGGT (SEQ ID NO:1) (accession number ss100070) and TGAACCTGGGAGGCGGAGCTTGCAGTGAGC(Y)GAGATCCCGCCACTGCACTC CAGCCTGGGC (SEQ ID NO:2) (accession number ss1000934)were found to represent regulatory SNP sequences and were included.

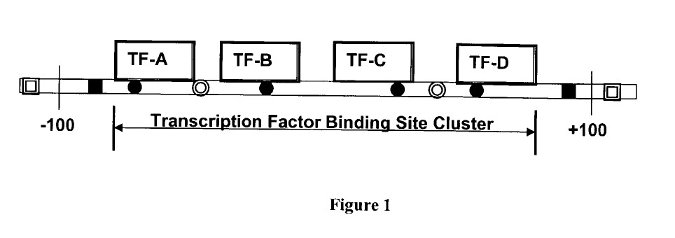

After completion of the comparisons, a query against the database was conducted to report the complete list of SNPs located within TFCs. Because in some instances, 100% sequence identity was found between more than one sequences using the 61 base pair search strings, the search was modified for those sequences to compare strings of 201 bases (with the SNP at the 101^{st} position) obtained from the flanking sequence in the SNP database. About 60,288 SNP sites were mapped, including about 33,441 regulatory SNPs located within transcription factor binding sites (“Core” regulatory SNPs), about 26,847 SNPs located within a cluster but between transcription factor binding sites, and about 18,263 SNPs located outside of transcription factor binding site clusters but within 100 bp 5′ and 100 bp 3′ to a transcription factor binding site cluster. FIG. 1 illustrates the relationship of SNPs to a Transcription Factor Binding Site Cluster. The map displays four hypothetical transcription factor binding sites (TF-A, TF-B, TF-C, and TF-D) comprising a hypothetical transcription factor binding site cluster within a stretch of genomic DNA. All four binding sites fall within a span of from 100 bases 5′ to the TFC to 100 bases 3′ to the TFC. Some SNPs, represented as filled circles, are within transcription factor binding sites. Other SNPs, represented by open circles, are within the cluster between neighboring transcription factor binding sites. Yet other SNPs, represented as filled squares, are within the span of the 100 bases 5 to 100 bases 3′ to the TFC but are outside the TFC. Still other SNPs, represented as open squares, may lie outside of the span.

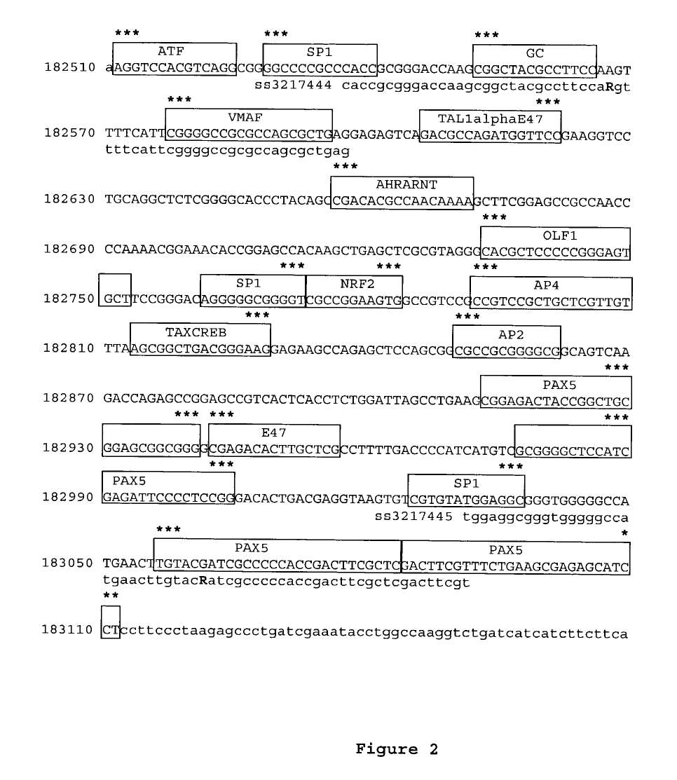

FIG. 2 illustrates the relationship of a TFC, the transcription factor binding sites within the TFC as well as two regulatory SNPs associated with the TFC. The sequence shown is nucleotide 182510 to nucleotide 183169 of clone AC025744.7. The part of that sequence which constitutes the TFC is shown in capital letters. Transcription factor binding site sequences are boxed in with the names of the transcription factors above each sequence. The asteriks above the boxes at the 5′ or 3′ ends of the transcription factor binding sites indicates the strand orientation of the consensus binding sequence for that factor. Two SNPs are found in the TFC region and these are shown in small letters beneath the TFC sequence as 30 nucleotides 5′ and 30 nucleotides 3′ to the SNP nucleotide which is capitalized and bolded. The accession number for each SNP precedes its sequence. SNP ss3217444 is not within a transcription factor binding site but falls in the TFC, whereas SNP ss3217445 falls within transcription binding factor site PAX5. The TFC shown in the figure is located within TSBP that is annotated in genbank under: NT_{—}028309.7 GI:22061729. That gene starts at 1,179,575 and goes up to 1,180,234. This TFC covers the region 1179575 to 1180175 and it is located on Chromosome 11.

The various groups were reported in U.S. Provisional Patent Application Nos. 60/332,723 and 60/334,543 filed Sep. 11, 2001 and Nov. 30, 2001, respectively. The tables in 60/334,543 report SNPs by accession number and by 41 bases of sequence, with the SNP site designated by a single letter in accordance with WIPO Standard ST.25 for nucleotides.

It is noted that the groups of regulatory SNPs reported earlier were generated using 41 nucleotides rather than 61 nucleotides as described above. The skilled artisan will appreciate that the database can be readily constructed using 61 nucleotides in the same manner as the databases illustrated earlier, which was generated using 41 nucleotides.

Table 1 of 60/334,543 shows the “Core” regulatory SNPs located within transcription factor binding sites. Clusters (represented by filled circles in FIG. 1). The table comprises 12,499 SNPs located within transcription factor binding sites.

Table 2 of 60/334,543 shows the regulatory SNPs located within TFCs, which includes “Core” regulatory SNPs plus SNPs located between identified transcription factor binding sites (the latter represented open circles in FIG. 1). The table comprises the 12,499 SNPs of table 1, plus 23,306 SNPs located within TFCs, but between adjacent transcription factor binding sites.

Table 3 of 60/334,543 shows the regulatory SNPs located within a TFC and flanking regions, including “Core” regulatory SNPs, plus SNPs located between transcription factor binding sites, plus 3,099 SNPs located outside of TFCs, but within 100 bp 5′ and 100 bp 3′ to a TFC (represented by filled squares in FIG. 1). Although not within TFCs, the last group of SNPs are potentially useful as genetic markers linked to transcription factor binding sites that relate to disease and they could also be located in transcription factor binding sites not yet identified..

All references and databases cited in this specification are hereby incorporated by reference. The discussion of the references herein is intended merely to summarize the assertions made by their authors and no admission is made that any of the references or statements therein constitute prior art relevant to patentability. Applicants reserve the right to challenge the accuracy and pertinency of the cited references.

What is claimed is:

1. A set of regulatory SNP polynucleotides or a set of polynucleotides complementary thereto, the set of regulatory SNP polynucleotides comprising a plurality of polynucleotides of at least 6 contiguous nucleotides, each polynucleotide of the set containing a regulatory SNP with 5′, 3′ or both 5′ and 3′ genomic flanking sequence, wherein the set of regulatory SNP polynucleotides contains a plurality of regulatory SNPs which collectively map to a plurality of TFC sequences such that each SNP lies within a TFC sequence and a genomic nucleic acid sequence from 30 nucleotides 5′ to 30 nucleotides 3′ to said each SNP is identical or complementary except for the SNP, to a portion of a genomic nucleic acid sequence from 30 nucleotides 5′ to 30 nucleotides 3′ to the TFC sequence.

2. A set according to claim 1 wherein the set of regulatory SNP polynucleotides comprises a plurality of polynucleotides of at least 10 contiguous nucleotides.

3. A set according to claim 1 wherein the TFC sequences are no longer than about 10 kb in length.

4. A set according to claim 1 wherein each of the TFC sequences comprise a sequence containing at least two transcription factor binding sites wherein likelihood of occurrence of the at least two transcription factor binding sites in the TFC sequence exceeds likelihood of occurrence in a random sequence.

5. A set according to claim 1 wherein each regulatory SNP when present in a genomic sequence, is associated with altered expression of a gene product as compared to expression of the same gene product from a genomic sequence containing a corresponding normal allele instead of the SNP.

6. A set according to claim 1 wherein each regulatory SNP has a higher likelihood of occurrence in a population of individuals having a disease or predisposition to having a disease as compared to occurrence in a population of individuals not having the disease or predisposition to having the disease.

7. A set according to claim 5 wherein each regulatory SNP has a higher likelihood of occurrence in a population of individuals having a disease or predisposition to having a disease as compared to occurrence in a population of individuals not having the disease or predisposition to having the disease.

8. A set according to claim 1 wherein the regulatory SNPs collectively map to a plurality of transcription factor binding site sequences, wherein each regulatory SNP lies within a transcription factor binding site sequence and a genomic sequence from 30 nucleotides 5′ to 30 nucleotides 3′ of said each SNP is identical or complementary except for the SNP, to a portion of a genomic nucleic acid sequence from 30 nucleotides 5′ to 30 nucleotides 3′ to said transcription factor binding site sequence.

9. A set according to claim 8 wherein each regulatory SNP when present in a genomic sequence, is associated with altered expression of a gene product as compared to expression of the same gene product from a genomic sequence containing a corresponding normal allele instead of the SNP.

10. A set according to claim 8 wherein each regulatory SNP has a higher likelihood of occurrence in a population of individuals having a disease or predisposition to having a disease as compared to occurrence in a population of individuals not having the disease or predisposition to having the disease.

11. A set according to claim 9 wherein each regulatory SNP has a higher likelihood of occurrence in a population of individuals having a disease or predisposition to having a disease as compared to occurrence in a population of individuals not having the disease or predisposition to having the disease.

12. A set according to claim 1 wherein each SNP lies within a genomic sequence from 100 nucleotides 5′ to 100 nucleotides 3′ to a TFC sequence and a genomic sequence from 30 nucleotides 5′ to 30 nucleotides 3′ of said each SNP is identical or complementary except for the SNP, to a portion of a genomic nucleic acid sequence from 130 nucleotides 5′ to 130 nucleotides 3′ to said at least one TFC sequence.

13. A set of probes or primers each of which comprises a polynucleotide of the set of regulatory SNP polynucleotides of claim 1 or the set of polynucleotides complementary thereto.

14. A set of probes or primers according to claim 13 wherein each probe consists of about 15 to about 30 contiguous nucleotides.

15. A biochip comprising the probes or primers of claim 13.

16. A set of probes or primers each of which comprises a polynucleotide of the set of regulatory SNP polynucleotides of claim 6 or the set of polynucleotides complementary thereto.

17. A set of probes or primers according to claim 16 wherein each probe consists of about 15 to about 30 contiguous nucleotides.

18. A biochip comprising the probes or primers of claim 16.

19. A set of probes or primers each of which comprises a polynucleotide of the set of regulatory SNP polynucleotides of claim 8 or the set of polynucleotides complementary thereto.

20. A set of probes according to claim 19 wherein each probe consists of about 15 to about 30 contiguous nucleotides.

21. A biochip comprising the probes of claim 19.

22. A set of probes or primers each of which comprises a polynucleotide of the set of regulatory SNP polynucleotides of claim 10 or the set of polynucleotides complementary thereto.

23. A set of probes according to claim 22 wherein each probe consists of about 15 to about 30 contiguous nucleotides.

24. A biochip comprising the probes of claim 22.

25. A method for diagnosing presence of a disease or predisposition for developing a disease in an individual, the method comprising determining whether a nucleic acid sample from the individual contains two or more regulatory SNP polynucleotides of a set of claim 5, wherein presence of the two or more regulatory SNP polynucleotides indicates presence of a disease or predisposition for developing a disease.

26. A method according to claim 25 wherein determining comprises determining whether the two or more regulatory SNP polynucleotides hybridize to the nucleic acid sample from the individual.

27. A method according to claim 26 wherein the two or more set of regulatory SNP polynucleotides are on a biochip.

28. A method for identifying a substance for treating a disease comprising testing a candidate compound for activity in modulating gene-product expression associated with a regulatory SNP polynucleotide of a set of regulatory SNP polynucleotides of claim 11.

29. A method of claim 28 wherein the gene product is overexpressed and the candidate compound is an antisense nucleic acid molecule comprising a complement of a genomic sequence which comprises the regulatory SNP polynucleotide.

30. A method of claim 28 wherein the gene product is underexpressed, wherein the regulatory SNP maps to a transcription factor binding site and wherein the candidate compound is a recombinant nucleic acid molecule which encodes a transcription factor which binds to the transcription factor binding site.

31. A method for treating or preventing a disease associated with a regulatory SNP in an individual, the method comprising administering to the individual a substance which modulates gene-product expression associated with a regulatory SNP polynucleotide of the set of regulatory SNP polynucleotide of claim 11.

32. A method of claim 31 wherein the gene product is overexpressed and the substance is an antisense nucleic acid molecule comprising a complement of the regulatory SNP polynucleotide.

33. A method of claim 31 wherein the gene product is underexpressed, wherein the regulatory SNP maps to a transcription factor binding site and wherein the substance is a recombinant nucleic acid molecule which encodes a transcription factor which binds to the transcription factor binding site.

34. A method of identifying regulatory SNPs, the method comprising determining that a SNP is a regulatory SNP if the SNP maps to a TFC wherein the SNP lies within a TFC sequence and a genomic nucleic acid sequence from at least 20 nucleotides 5′ to at least 20 nucleotides 3′ to said candidate SNP is identical or complementary except for the SNP, to a portion of a genomic nucleic acid sequence from at least 20 nucleotides 5′ to at least 20 nucleotides 3′ to the TFC sequence.

35. A method according to claim 34 wherein determining comprises determining that a SNP is a regulatory SNP if the SNP lies within a TFC sequence and a genomic nucleic acid sequence from at least 30 nucleotides 5′ to at least 30 nucleotides 3′ to said candidate SNP is identical or complementary except for the SNP, to a portion of a genomic nucleic acid sequence from at least 30 nucleotides 5′ to at least 30 nucleotides 3′ to the TFC sequence.

36. A method according to claim 34 wherein the TFC sequence is no longer than about 10 kb in length.

37. A method according to claim 34 wherein the TFC sequence contains at least two transcription factor binding sites wherein the likelihood of occurrence of the at least two transcription factor binding sites in the TFC sequence exceeds likelihood of occurrence in a random sequence.

38. A method according to claim 34 wherein the regulatory SNP when present in a genomic sequence, is associated with an altered expression of a gene product as compared to expression of the same gene product from a genomic sequence containing a corresponding normal allele instead of the SNP.

39. A method according to claim 34 wherein the regulatory SNP has a higher likelihood of occurrence in a population of individuals having a disease or predisposition to having a disease as compared to occurrence in a population of individuals not having the disease or predisposition to having the disease.

40. A method according to claim 37 wherein the regulatory SNP has a higher likelihood of occurrence in a population of individuals having a disease or predisposition to having a disease as compared to occurrence a population of individuals not having the disease or predisposition to having the disease.

41. A method according to claim 34 wherein determining comprises determining that a SNP is a regulatory SNP if the SNP maps to a transcription factor binding site, wherein the SNP lies within a transcription factor binding site sequence and a genomic sequence from 20 nucleotides 5′ to 20 nucleotides 3′ of said each SNP is identical or complementary except for the SNP, to a portion of a genomic nucleic acid sequence from 20 nucleotides 5′ to 20 nucleotides 3′ to said transcription factor binding site sequence.

42. A method according to claim 34 wherein determining comprises determining that a SNP is a regulatory SNP if the SNP maps to a transcription factor binding site, wherein the SNP lies within a transcription factor binding site sequence and a genomic sequence from 30 nucleotides 5′ to 30 nucleotides 3′ of said each SNP is identical or complementary except for the SNP, to a portion of a genomic nucleic acid sequence from 30 nucleotides 5′ to 30 nucleotides 3′ to said transcription factor binding site sequence.

43. A method according to claim 42 wherein the regulatory SNP is associated with an altered expression of a gene product as compared to expression of the same gene product from a genomic sequence containing a corresponding normal allele.

44. A method according to claim 41 wherein the regulatory SNP has a higher likelihood of occurrence in a population of individuals having a disease or predisposition to having a disease as compared to occurrence in a population of individuals not having the disease or predisposition to having the disease.

45. A method according to claim 43 wherein the regulatory SNP has a higher likelihood of occurrence in a population of individuals having a disease or predisposition to having a disease as compared to occurrence a population of individuals not having the disease or predisposition to having the disease.

46. A method according to claim 34 wherein determining comprises determining that a SNP is a regulatory SNP if the SNP lies within a genomic sequence from 100 nucleotides 5′ to 100 nucleotides 3′ to a TFC sequence and a genomic sequence from 30 nucleotides 5′ to 30 nucleotides 3′ of the SNP is identical or complementary except for the SNP, to a portion of a genomic nucleic acid sequence from 130 nucleotides 5′ to 130 nucleotides 3′ to the TFC sequence.

47. A method of generating a set of probes for use in detecting presence of regulatory SNPs, the method comprising identifying a set two or more regulatory SNPs which map to TFCs in accordance with a method of claim 34, identifying 5′ genomic flanking sequence, 3′ genomic flanking sequence or both 5′ and 3′ genomic flanking sequence, and preparing said probes.

48. A method according to claim 47 wherein each probe consists of about 15 to about 30 contiguous nucleotides.

49. A method of constructing a biochip for detecting presence of regulatory SNPs, the method comprising generating the probes of claim 47 and placing the probes on the biochip.

50. A method of generating a set of probes for use in identifying regulatory SNPs, the method comprising identifying a set two or more regulatory SNPs which map to TFCs in accordance with claim 41, identifying 5′ genomic flanking sequence, 3′ genomic flanking sequence or both 5′ and 3′ genomic flanking sequence, and preparing said probes.

51. A method according to claim 50 wherein each probe consists of about 15 to about 30 contiguous nucleotides.

52. A method of constructing a biochip for detecting presence of regulatory SNPs, the method comprising generating the probes of claim 50 and placing the probes on the biochip.

53. A computer readable medium having a data structure for use in reporting regulatory SNPs, the data structure comprising a first field containing either or both of sequence and genomic mapping location sequence information on SNPs; a second field containing either or both of sequence and genomic mapping location of TFCs; and a third field containing information on regulatory SNPs, the SNPs being identified by identities of either or both of sequences and genomic mapping locations of SNPs and TFCs.

54. A computer readable medium according to claim 53, wherein the first field containing nucleic acid sequence information on nucleic acid molecules which contain a SNP; a second field containing nucleic acid sequence information on TFC sequences; and a third field containing information on regulatory SNPs, the regulatory SNPs being identified by sequence identities of nucleic acid sequences containing SNPs and TFC sequences.

55. A computer readable medium of claim 53 wherein the nucleic acid sequence information on TFCs comprises nucleic acid sequence information on transcription factor binding sites and wherein the information on regulatory SNPs comprises identification of the regulatory SNPs by sequence identity of nucleic acid sequences containing SNPs and transcription factor binding site sequences.

56. A computer readable medium of claim 52 wherein the third field comprises information identifying the regulatory SNP and flanking sequence, the regulatory SNP being within a TFC sequence and a genomic nucleic acid sequence from 30 nucleotides 5′ to 30 nucleotides 3′ to said the SNP being identical except for the SNP, to a portion of a genomic nucleic acid sequence from 30 nucleotides 5′ to 30 nucleotides 3′ to the TFC sequence.
